(19) United States
(12) Patent Application Publication
(10) Pub. No.: US 2023/0118869 A1
(43) Pub. Date: Apr. 20, 2023

(54) COMPOSITION FOR PREVENTING OR TREATING ALZHEIMER'S DISEASE COMPRISING PHOSPHOLIPASE C ACTIVATOR AS AN ACTIVE INGREDIENT

(71) Applicant: Korea University Research and Business Foundation, Seoul (KR)

(72) Inventors: Jeehyun KWAG, Seoul (KR); Jaedong LEE, Seoul (KR)

(73) Assignee: Korea University Research and Business Foundation, Seoul (KR)

(21) Appl. No.: 17/965,941

(22) Filed: Oct. 14, 2022

(30) Foreign Application Priority Data
Oct. 15, 2021 (KR) 10-2021-0137527
Mar. 18, 2022 (KR) 10-2022-0034260

(51) Int. Cl. A61K 31/18; G01N 33/68; A61P 25/28

(52) U.S. Cl. CPC A61K 31/18; A61P 25/28; G01N 33/6896; G01N 2333/916; G01N 2800/2821

(57) ABSTRACT

The present invention relates to a composition for preventing or treating Alzheimer’s disease, comprising a phospholipase C (PLC) activator as an active ingredient. A composition comprising the PLC activator of the present invention as an active ingredient restores the S-eCB mobilization suppressed by AβO, recovers the synaptic plasticity impaired by AβO, and not only recovers PLCβ1 protein levels to normal levels in AβO-treated mouse hippocampal slices and 5XFAD mouse hippocampal slices in the chronic stage of AD, but also recovers contextual fear memory impairment in AD mice, and thus is expected to be usefully used for preventing or treating Alzheimer’s disease.

### CROSS REFERENCE TO RELATED APPLICATION

This application claims priority to and the benefit of Korean Patent Application No. 10-2021-0137527, filed on Oct. 15, 2021, and No. 10-2022-0034260, filed on Mar. 18, 2022, the disclosure of which is incorporated herein by reference in its entirety.

### BACKGROUND

### 1. Field of the Invention

The present invention relates to a composition for preventing or treating Alzheimer’s disease, comprising a phospholipase C (PLC) activator as an active ingredient.

### 2. Discussion of Related Art

Dementia is a pathological phenomenon that should be distinguished from normal aging, and is classified into Alzheimer’s disease, vascular dementia, and other dementia due to alcoholism, trauma, or sequelae of Parkinson’s disease according to its cause.

Alzheimer’s disease is a degenerative brain disease that mainly affects elderly people aged 65 and over and is characterized by gradual memory loss and cognitive impairment. At the pathological level, the tangle phenomenon of betaamyloid protein deposits and nerve fiber tau proteins is a typical etiology. Alzheimer’s disease is often accompanied by psychobehavioral symptoms such as personality changes, agitated behavior, depression, delusions, hallucinations, increased aggression, and sleep disorders, as well as cognitive decline during its progression, and at the end of Alzheimer’s disease, neurological disorders such as stiffness and gait abnormalities, as well as physical complications such as bowel and bladder incontinence, infection, and bedsore appear. When the brain tissue of Alzheimer’s disease patients is examined under a microscope, characteristic lesions such as neuritic plaques and neurofibrillary tangles are observed, and general brain atrophy findings due to neuronal loss are seen during macroscopic observation. Such brain pathological findings appear only in the hippocampus and entorhinal cortex regions, which are the main brain regions responsible for memory, in the early stage of the disease, but gradually spread from the parietal lobe, frontal lobe, and the like to the entire brain. According to the progression of cerebral pathological infringement sites, memory loss mainly appears in the early stage, and as the disease progresses, clinical symptoms are diversified and become more severe while showing a gradual course.

Although the number of patients with Alzheimer’s disease due to the aging population is significantly increasing and the disease is rapidly emerging as a social problem to be solved, there are almost no drugs capable of effectively preventing or treating the disease. Dementia is a neurological disease that afflicts 10% or more of the elderly population aged 65 and over, 50% or more of dementias are reported to be Alzheimer’s disease, and in 2017, the elderly population ratio exceeded 7 million, which is 14% of the total population, and is expected to rapidly increase to 41.0% in 2060. Further, the number of dementia patients has reached 724,000 as of 2017, and is expected to increase to 1.68 million in 2040 and 2.127 million or more in 2050 as the population ages.

However, the current therapeutic methods for dementia are measures not for solving the fundamental cause, but merely for the purpose of alleviating the symptoms, and there is a need for developing a material for suppressing, delaying, or treating the onset of Alzheimer’s disease.

### SUMMARY OF THE INVENTION

As a result of research for the development of an agent for preventing or treating Alzheimer’s disease, the present inventors confirmed a mechanism by which an amyloid beta oligomer (AβO) suppresses synergistic enhancement of endocannabinoid (S-eCB) mobilization by reducing PLCβ, and confirmed that a phospholipase C (PLC) activator of the present invention restored the S-eCB mobilization suppressed by AβO, recovered the spike timing-dependent long-term potentiation (tLTP) of synaptic plasticity in the hippocampus impaired by AβO, and not only recovered PLCβ1 protein levels to normal levels in AβO-treated mouse hippocampal slices and 5XFAD mouse hippocampal slices in the chronic stage of AD, but also recovered contextual fear memory impairment in AD mice, thereby completing the present invention based on this.

Thus, an object of the present invention is to provide a pharmaceutical composition for preventing or treating Alzheimer’s disease, comprising a phospholipase C (PLC) activator as an active ingredient.

Another object of the present invention is to provide a method for screening a therapeutic agent for Alzheimer’s disease, the method comprising the following steps:
(a) treating a biological sample isolated from a patient with Alzheimer’s disease with a candidate material;
(b) measuring the activity level of PLCβ1 protein or the synthesis or secretion level of eCB in the sample; and
(c) determining the candidate material as a therapeutic agent for Alzheimer’s disease when the activity level of PLCβ1 protein or the synthesis or secretion level of eCB is increased compared to a group untreated with the candidate material.

Still another object of the present invention is to provide an information providing method for diagnosing Alzheimer’s disease, the method comprising measuring the activity level of PLCβ1 protein in a biological sample isolated from a subject.

However, the technical problems which the present invention intends to solve are not limited to the technical problems that have been mentioned above, and other technical problems which have not been mentioned will be clearly understood by a person with ordinary skill in the art to which the present invention pertains from the following description.

To achieve the objects of the present invention, the present invention provides a pharmaceutical composition for preventing or treating Alzheimer’s disease, comprising a phospholipase C (PLC) activator as an active ingredient.

Further, the present invention provides a method for screening a therapeutic agent for Alzheimer’s disease, the method comprising the following steps:
(a) treating a biological sample isolated from a patient with Alzheimer’s disease with a candidate material;
(b) measuring the activity level of PLCβ1 protein or the synthesis or secretion level of eCB in the sample; and
(c) determining the candidate material as a therapeutic agent for Alzheimer’s disease when the activity level of PLCβ1 protein or the synthesis or secretion level of eCB is increased compared to a group untreated with the candidate material.

In addition, the present invention provides an information providing method for diagnosing Alzheimer’s disease, the method comprising measuring the activity level of PLCβ1 protein in a biological sample isolated from a subject.

In addition, the present invention provides a method for diagnosing Alzheimer’s disease, the method comprising measuring the activity level of PLCβ1 protein in a biological sample isolated from a subject.

In an exemplary embodiment of the present invention, the PLC activator may be characterized by increasing the activity of PLCβ1 protein, but is not limited thereto.

In another exemplary embodiment of the present invention, the PLC activator may be characterized by increasing the synthesis and secretion of endocannabinoid (eCB), but is not limited thereto.

In still another exemplary embodiment of the present invention, the PLC activator may be characterized by recovering any one or more of the following characteristics, but is not limited thereto:
(a) hippocampal synaptic plasticity; and
(b) contextual fear memory.

In yet another exemplary embodiment of the present invention, the PLC activator may be characterized by being m-3M3FBS, but is not limited thereto.

In yet another exemplary embodiment of the present invention, the sample may be characterized by being a hippocampus-derived sample, but is not limited thereto.

In yet another exemplary embodiment of the present invention, the measurement in Step (b) may be characterized by being performed by one or more methods selected from the group consisting of western blot, enzyme linked immunosorbent assay (ELISA), radioimmunoassay (RIA), radioimmunodiffusion, and immunoprecipitation assay, but the method is not limited thereto.

In yet another exemplary embodiment of the present invention, the subject may be characterized by being a mammal comprising a human, but is not limited thereto.

In yet another exemplary embodiment of the present invention, the method may be characterized by further comprising determining, as Alzheimer’s disease, the case where the activity level of the PLCβ1 protein is reduced compared to the activity level of the PLCβ1 protein of a control, but is not limited thereto.

In yet another exemplary embodiment of the present invention, the activity level of PLCβ1 protein may be characterized by being measured by one or more methods selected from the group consisting of western blot, enzyme linked immunosorbent assay (ELISA), radioimmunoassay (RIA), radioimmunodiffusion, and immunoprecipitation assay, but the method is not limited thereto.

In addition, the present invention provides a method for preventing or treating Alzheimer’s disease, the method comprising administering a composition comprising a phospholipase C (PLC) activator as an active ingredient to a subject in need.

Furthermore, the present invention provides a use of a composition comprising a PLC activator as an active ingredient for preventing or treating Alzheimer’s disease.

Further, the present invention provides a use of a PLC activator for preparing a drug for treating Alzheimer’s disease.

### BRIEF DESCRIPTION OF THE DRAWINGS

The above and other objects, features and advantages of the present invention will become more apparent to those of ordinary skill in the art by describing in detail exemplary embodiments thereof with reference to the accompanying drawings, in which:

FIG. 1A illustrates an S-eCB mobilization protocol;

FIGS. 1B to 1D illustrate the results of analyzing eIPSC amplitudes with or without a CB1R antagonist AM251 under the Group 1 mGluR agonist DHPG or DHPG + CA1PC (pyramidal cell) neural spike conditions;

FIGS. 1E to 1H illustrates the results of treating DMSO-treated, AβO-treated, and scrambled AβO-treated rat hippocampal slices with an mGluR1 antagonist LY367385 (100 µM) or an mGluR5 antagonist MPEP (10 µM) and analyzing eIPSC amplitudes;

FIGS. 2A and 2B illustrates the results of treating DMSO-treated, AβO-treated, and scrambled AβO-treated rat hippocampal slices with a PLCβ blocker U73122 (5 µM) and analyzing eIPSC amplitudes;

FIGS. 2C and 2D illustrate the results of treating DMSO-treated, AβO-treated, and scrambled AβO-treated rat hippocampal slices with a PLCβ activator m-3M3FBS (30 µM) and analyzing eIPSC amplitudes;

FIGS. 2E and 2F illustrate the results of analyzing the protein expression levels of PLCβ1 in DMSO-treated, AβO-treated, and AβO + m-3M3FBS-treated rat hippocampal slices;

FIGS. 3A and 3B illustrate the schematic views of an S-eCB mobilization-ensuring tLTP protocol;

FIG. 3C illustrates the results of inducing tLTP, and then analyzing EPSP slopes in DMSO-treated rat hippocampal slices;

FIG. 3D illustrates the results of treating DMSO-treated rat hippocampal slices with D-AP5 (50 µM), inducing tLTP, and then analyzing EPSP slopes;

FIG. 3E illustrates the results of treating DMSO-treated rat hippocampal slices with AM251 (3 µM), inducing tLTP, and then analyzing EPSP slopes;

FIG. 3F illustrates the results of inducing tLTP, and then analyzing EPSP slopes in AβO-treated rat hippocampal slices;

FIG. 3G illustrates the results of treating AβO-treated rat hippocampal slices with m-3M3FBS (30 µM), inducing tLTP, and then analyzing EPSP slopes;

FIG. 3H illustrates a comparative analysis of the EPSP slopes analyzed in FIGS. 3C to 3G;

FIG. 4A illustrates a schematic view of injecting m-3M3FBS into both the dorsal and ventral regions of the bilateral hippocampus of 5XFAD mice;

FIGS. 4B and 4C illustrate the results of confirming the activity of PLCβ1 protein in the hippocampal slices of WT mice, 5XFAD mice, and m-3M3FBSinjected 5XFAD mice by western blot;

FIG. 4D illustrates a schematic view of a contextual fear conditioning experiment which is a hippocampus-dependent fear memory experiment for memory performance-related behavioral experiments;

FIG. 4E analyzes the degree of freezing in WT mice, 5XFAD mice, and m-3M3FBS-injected 5XFAD mice; and

FIG. 5 illustrates a schematic view of the recovery mechanism of synaptic plasticity impairment and memory ability disorder by PLC activators in the hippocampus of Alzheimer’s disease.

### DETAILED DESCRIPTION OF EXEMPLARY EMBODIMENTS

In an exemplary embodiment of the present invention, it was confirmed that the activation of Group 1 mGluRs induced eCB mobilization, an in vivo analogous neural spike train induced S-eCB mobilization during Group 1 mGluR activation, and the S-eCB mobilization was specifically impaired by AβO (see Example 1).

In another exemplary embodiment of the present invention, it was confirmed that AβO interfered with the PLCβ pathway to block S-eCB mobilization in CA1 PC, and the S-eCB mobilization suppressed by AβO was restored when PLC activation was increased using m-3M3FBS (see Example 2).

In still another exemplary embodiment of the present invention, it was confirmed that AβO completely blocked the induction of spike timing-dependent long-term potentiation (tLTP), and it was confirmed that when the PLCβ-dependent S-eCB mobilization was improved by directly increasing PLCβ1 protein levels, hippocampal tLTP impaired by AβO was recovered (see Example 3).

In yet another exemplary embodiment of the present invention, it was confirmed that m-3M3FBS completely recovered PLCβ1 protein levels to normal levels in AβO-treated mouse hippocampal slices and 5XFAD mouse hippocampal slices in the chronic stage of AD, and it was confirmed that contextual fear memory impairment was recovered in AD mice (see Example 4).

Hereinafter, the present invention will be described in detail.

The present invention provides a pharmaceutical composition for preventing or treating Alzheimer’s disease, comprising a phospholipase C (PLC) activator as an active ingredient.

In addition, the present invention provides a method for preventing or treating Alzheimer’s disease, the method comprising administering a composition comprising a phospholipase (PLC) activator as an active ingredient to a subject in need.

Furthermore, the present invention provides a use of a composition comprising a PLC activator as an active ingredient for preventing or treating Alzheimer’s disease.

Further, the present invention provides a use of a PLC activator for preparing a drug for treating Alzheimer’s disease.

In the present invention, the PLC activator may increase the activity of PLCβ1 protein or increase the synthesis and secretion of endocannabinoid (eCB), but is not limited thereto. In addition, the PLC activator may recover any one or more of the following characteristics, but is not limited thereto: (a) hippocampal synaptic plasticity; and (b) contextual fear memory.

As used herein, “phospholipase C (PLC)” refers to a very important enzyme for intracellular signal transduction, and is activated by signals received through various hormones and growth factor receptors. A stimulated PLC increases intracellular calcium concentration through IP₃ and DAG, respectively, and activates protein kinase C to activate enzymes related to various intracellular signaling systems. The PLC may be PLCβ, PLCγ, PLCδ6, PLCε, PLCζ, or PLCη, but is not limited thereto.

In the present invention, the PLC activator is not limited as long as it is a material that increases the activity of PLC, and may be m-3M3FBS, Spermine, Pseudolaric acid B, or O-3M3FBS, but is not limited thereto. Furthermore, the PLC activator may be comprised at a concentration of 1 to 100 µM, 1 to 70 µM, 1 to 40 µM, 10 to 100 µM, 10 to 70 µM, 10 to 40 µM, 20 to 100 µM, 20 to 70 µM, or 20 to 40 µM in the entire composition, but the concentration is not limited thereto.

In the present invention, the m-3M3FBS may be represented by the following Chemical Formula 1, the Spermine may be represented by the following Chemical Formula 2, the Pseudolaric acid B may be represented by the following Chemical Formula 3, and the O-3M3FBS may be represented by the following Chemical Formula 4, but are not limited thereto.

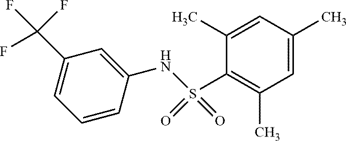 [SMILES: Cc1cc(C)c(S(=O)(=O)Nc2cccc(C(F)(F)F)c2)c(C)c1]

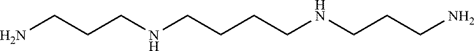 [SMILES: NCCCNCCCCNCCCN]

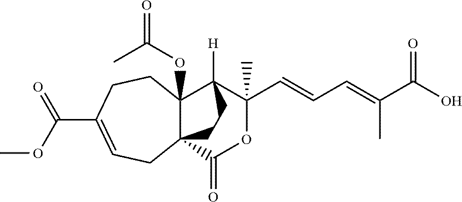 [SMILES: COC(=O)C1=CC[C@@]23CC[C@@H]([C@@](C)(/C=C/C=C(\C)C(=O)O)OC2=O)[C@@]3(OC(C)=O)CC1]

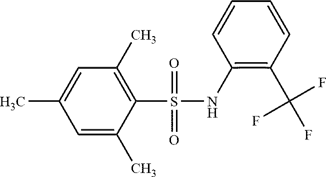 [SMILES: Cc1cc(C)c(S(=O)(=O)Nc2ccccc2C(F)(F)F)c(C)c1]

As used herein, “Alzheimer’s disease” refers to a neurodegenerative disease that causes disorders in all cognitive functions including memory functions, which are caused by the death of neurons and the disruption of neural circuits as an amyloid beta peptide (Aβ) is deposited at interneuronal synapses in various brain regions including the hippocampus. Abnormal deposition of Aβ in the hippocampal region of the brain reduces the long-term synaptic plasticity phenomenon, which is known as the biological mechanism of memory. The eCB is a retrograde neurotransmitter, and plays an important role in inducing hippocampal synaptic plasticity.

As used herein, “synaptic plasticity” means that synapses respond to an increase or decrease in activity, and is strengthened or weakened over time. Since memory relies on extensive neurotransmission in the brain, synaptic plasticity plays an important role in learning and memory. In an exemplary embodiment of the present invention, synaptic plasticity was confirmed through a spike timing-synapse enhancement (timing-dependent potentiation, tLTP) protocol, but is not limited thereto.

As used herein, “recovery” refers to returning to a normal state or regaining a normal state. In an exemplary embodiment of the present invention, recovery may mean not developing Alzheimer’s disease or returning to the same state as a control in which AβO was not treated, but is not limited thereto.

The present invention may also comprise a pharmaceutically acceptable salt of a PLC activator as an active ingredient. In the present invention, the term “pharmaceutically acceptable salt” includes a salt derived from a pharmaceutically acceptable inorganic acid, organic acid, or base.

Examples of a suitable acid include hydrochloric acid, bromic acid, sulfuric acid, nitric acid, perchloric acid, fumaric acid, maleic acid, phosphoric acid, glycolic acid, lactic acid, salicylic acid, succinic acid, toluene-p-sulfonic acid, tartaric acid, acetic acid, citric acid, methanesulfonic acid, formic acid, benzoic acid, malonic acid, gluconic acid, naphthalene-2-sulfonic acid, benzenesulfonic acid, and the like. An acid addition salt may be prepared by a typical method, for example, dissolving a compound in an excessive amount of an aqueous acid solution and precipitating the salt using a water-miscible organic solvent such as methanol, ethanol, acetone or acetonitrile. In addition, the acid addition salt may be prepared by heating the same molar amount of compound and an acid or alcohol in water, subsequently evaporating the mixture to dry the mixture, or suction-filtering the precipitated salt.

A salt derived from a suitable base may include an alkali metal such as sodium and potassium, an alkaline earth metal such as magnesium, ammonium and the like, but is not limited thereto. An alkali metal or alkaline earth metal salt may be obtained by, for example, dissolving the compound in an excessive amount of an alkali metal hydroxide or alkaline earth metal hydroxide solution, filtering a nonsoluble compound salt, evaporating the filtrate, and drying the resulting product. In this case, it is particularly suitable to prepare a sodium, potassium or calcium salt as the metal salt from the pharmaceutical perspective, and the corresponding silver salt may also be obtained by reacting an alkali metal or alkaline earth metal salt with a suitable silver salt (for example, silver nitrate).

The amount of the PLC activator in the composition of the present invention may be appropriately adjusted depending on the symptoms of a disease, the degree of progression of symptoms, the condition of a patient, and the like, and may range from, for example, 0.0001 wt% to 99.9 wt% or 0.001 wt% to 50 wt% with respect to a total weight of the composition, but the present invention is not limited thereto. The amount ratio is a value based on the amount of dried product from which a solvent is removed.

The pharmaceutical composition according to the present invention may further include a suitable carrier, excipient, and diluent which are commonly used in the preparation of pharmaceutical compositions. The excipient may be, for example, one or more selected from the group consisting of a diluent, a binder, a disintegrant, a lubricant, an adsorbent, a humectant, a film-coating material, and a controlled release additive.

The pharmaceutical composition according to the present invention may be used by being formulated, according to commonly used methods, into a form such as powders, granules, sustained-release-type granules, enteric granules, liquids, eye drops, elixirs, emulsions, suspensions, spirits, troches, aromatic water, lemonades, tablets, sustained-release-type tablets, enteric tablets, sublingual tablets, hard capsules, soft capsules, sustained-release-type capsules, enteric capsules, pills, tinctures, soft extracts, dry extracts, fluid extracts, injections, capsules, perfusates, or a preparation for external use, such as plasters, lotions, pastes, sprays, inhalants, patches, sterile injectable solutions, or aerosols. The preparation for external use may have a formulation such as creams, gels, patches, sprays, ointments, plasters, lotions, liniments, pastes, or cataplasmas.

As the carrier, the excipient, and the diluent that may be included in the pharmaceutical composition according to the present invention, lactose, dextrose, sucrose, oligosaccharides, sorbitol, mannitol, xylitol, erythritol, maltitol, starch, acacia rubber, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate, and mineral oil may be used.

For formulation, commonly used diluents or excipients such as fillers, thickeners, binders, wetting agents, disintegrants, and surfactants are used.

As additives of tablets, powders, granules, capsules, pills, and troches according to the present invention, excipients such as corn starch, potato starch, wheat starch, lactose, white sugar, glucose, fructose, D-mannitol, precipitated calcium carbonate, synthetic aluminum silicate, dibasic calcium phosphate, calcium sulfate, sodium chloride, sodium hydrogen carbonate, purified lanolin, microcrystalline cellulose, dextrin, sodium alginate, methyl cellulose, sodium carboxymethylcellulose, kaolin, urea, colloidal silica gel, hydroxypropyl starch, hydroxypropyl methylcellulose (HPMC), HPMC 1928, HPMC 2208, HPMC 2906, HPMC 2910, propylene glycol, casein, calcium lactate, and Primojel®; and binders such as gelatin, Arabic gum, ethanol, agar powder, cellulose acetate phthalate, carboxymethylcellulose, calcium carboxymethylcellulose, glucose, purified water, sodium caseinate, glycerin, stearic acid, sodium carboxymethylcellulose, sodium methylcellulose, methylcellulose, microcrystalline cellulose, dextrin, hydroxycellulose, hydroxypropyl starch, hydroxymethylcellulose, purified shellac, starch, hydroxypropyl cellulose, hydroxypropyl methylcellulose, polyvinyl alcohol, and polyvinylpyrrolidone may be used, and disintegrants such as hydroxypropyl methylcellulose, corn starch, agar powder, methylcellulose, bentonite, hydroxypropyl starch, sodium carboxymethylcellulose, sodium alginate, calcium carboxymethylcellulose, calcium citrate, sodium lauryl sulfate, silicic anhydride, 1-hydroxypropylcellulose, dextran, ion-exchange resin, polyvinyl acetate, formaldehyde-treated casein and gelatin, alginic acid, amylose, guar gum, sodium bicarbonate, polyvinylpyrrolidone, calcium phosphate, gelled starch, Arabic gum, amylopectin, pectin, sodium polyphosphate, ethyl cellulose, white sugar, magnesium aluminum silicate, a di-sorbitol solution, and light anhydrous silicic acid; and lubricants such as calcium stearate, magnesium stearate, stearic acid, hydrogenated vegetable oil, talc, lycopodium powder, kaolin, Vaseline, sodium stearate, cacao butter, sodium salicylate, magnesium salicylate, polyethylene glycol (PEG) 4000, PEG 6000, liquid paraffin, hydrogenated soybean oil (Lubri wax), aluminum stearate, zinc stearate, sodium lauryl sulfate, magnesium oxide, Macrogol, synthetic aluminum silicate, silicic anhydride, higher fatty acids, higher alcohols, silicone oil, paraffin oil, polyethylene glycol fatty acid ether, starch, sodium chloride, sodium acetate, sodium oleate, dl-leucine, and light anhydrous silicic acid may be used.

As additives of liquids according to the present invention, water, dilute hydrochloric acid, dilute sulfuric acid, sodium citrate, monostearic acid sucrose, polyoxyethylene sorbitol fatty acid esters (twin esters), polyoxyethylene monoalkyl ethers, lanolin ethers, lanolin esters, acetic acid, hydrochloric acid, ammonia water, ammonium carbonate, potassium hydroxide, sodium hydroxide, prolamine, polyvinylpyrrolidone, ethylcellulose, and sodium carboxymethylcellulose may be used.

In syrups according to the present invention, a white sugar solution, other sugars or sweeteners, and the like may be used, and as necessary, a fragrance, a colorant, a preservative, a stabilizer, a suspending agent, an emulsifier, a viscous agent, or the like may be used.

In emulsions according to the present invention, purified water may be used, and as necessary, an emulsifier, a preservative, a stabilizer, a fragrance, or the like may be used.

In suspensions according to the present invention, suspending agents such as acacia, tragacanth, methylcellulose, carboxymethylcellulose, sodium carboxymethylcellulose, microcrystalline cellulose, sodium alginate, hydroxypropyl methylcellulose (HPMC), HPMC 1828, HPMC 2906, HPMC 2910, and the like may be used, and as necessary, a surfactant, a preservative, a stabilizer, a colorant, and a fragrance may be used.

Injections according to the present invention may include: solvents such as distilled water for injection, a 0.9% sodium chloride solution, Ringer’s solution, a dextrose solution, a dextrose+sodium chloride solution, PEG, lactated Ringer’s solution, ethanol, propylene glycol, non-volatile oil-sesame oil, cottonseed oil, peanut oil, soybean oil, corn oil, ethyl oleate, isopropyl myristate, and benzene benzoate; cosolvents such as sodium benzoate, sodium salicylate, sodium acetate, urea, urethane, monoethylacetamide, butazolidine, propylene glycol, the Tween series, amide nicotinate, hexamine, and dimethylacetamide; buffers such as weak acids and salts thereof (acetic acid and sodium acetate), weak bases and salts thereof (ammonia and ammonium acetate), organic compounds, proteins, albumin, peptone, and gums; isotonic agents such as sodium chloride; stabilizers such as sodium bisulfite (NaHSO₃) carbon dioxide gas, sodium metabisulfite (Na₂S₂O₅), sodium sulfite (Na₂SO₃), nitrogen gas (N₂), and ethylenediamine tetraacetic acid; sulfating agents such as 0.1% sodium bisulfide, sodium formaldehyde sulfoxylate, thiourea, disodium ethylenediaminetetraacetate, and acetone sodium bisulfite; a pain relief agent such as benzyl alcohol, chlorobutanol, procaine hydrochloride, glucose, and calcium gluconate; and suspending agents such as sodium CMC, sodium alginate, Tween 80, and aluminum monostearate.

In suppositories according to the present invention, bases such as cacao butter, lanolin, Witepsol, polyethylene glycol, glycerogelatin, methylcellulose, carboxymethylcellulose, a mixture of stearic acid and oleic acid, Subanal, cottonseed oil, peanut oil, palm oil, cacao butter + cholesterol, lecithin, lanette wax, glycerol monostearate, Tween or span, imhausen, monolan(propylene glycol monostearate), glycerin, Adeps solidus, buytyrum Tego-G, cebes Pharma 16, hexalide base 95, cotomar, Hydrokote SP, S-70-XXA, S-70-XX75(S-70-XX95), Hydrokote 25, Hydrokote 711, idropostal, massa estrarium (A, AS, B, C, D, E, I, T), masa-MF, masupol, masupol-15, neosuppostal-N, paramount-B, supposiro OSI, OSIX, A, B, C, D, H, L, suppository base IV types AB, B, A, BC, BBG, E, BGF, C, D, 299, suppostal N, Es, Wecoby W, R, S, M, Fs, and tegester triglyceride matter (TG-95, MA, 57) may be used.

Solid preparations for oral administration include tablets, pills, powders, granules, capsules, and the like, and such solid preparations are formulated by mixing the composition with at least one excipient, e.g., starch, calcium carbonate, sucrose, lactose, gelatin, and the like. In addition to simple excipients, lubricants such as magnesium stearate and talc are also used.

Examples of liquid preparations for oral administration include suspensions, liquids for internal use, emulsions, syrups, and the like, and these liquid preparations may include, in addition to simple commonly used diluents, such as water and liquid paraffin, various types of excipients, for example, a wetting agent, a sweetener, a fragrance, a preservative, and the like. Preparations for parenteral administration include an aqueous sterile solution, a non-aqueous solvent, a suspension, an emulsion, a freeze-dried preparation, and a suppository. Non-limiting examples of the non-aqueous solvent and the suspension include propylene glycol, polyethylene glycol, a vegetable oil such as olive oil, and an injectable ester such as ethyl oleate.

The pharmaceutical composition according to the present invention is administered in a pharmaceutically effective amount. In the present invention, “the pharmaceutically effective amount” refers to an amount sufficient to treat diseases at a reasonable benefit/risk ratio applicable to medical treatment, and an effective dosage level may be determined according to factors including types of diseases of patients, the severity of disease, the activity of drugs, sensitivity to drugs, administration time, administration route, excretion rate, treatment period, and simultaneously used drugs, and factors well known in other medical fields.

The composition according to the present invention may be administered as an individual therapeutic agent or in combination with other therapeutic agents, may be administered sequentially or simultaneously with therapeutic agents in the related art, and may be administered in a single dose or multiple doses. It is important to administer the composition in a minimum amount that can obtain the maximum effect without any side effects, in consideration of all the aforementioned factors, and this may be easily determined by those of ordinary skill in the art.

The pharmaceutical composition of the present invention may be administered to a subject via various routes. All administration methods can be predicted, and the pharmaceutical composition may be administered via, for example, oral administration, subcutaneous injection, intraperitoneal injection, intravenous injection, intramuscular injection, intrathecal (space around the spinal cord) injection, sublingual administration, administration via the buccal mucosa, intrarectal insertion, intravaginal insertion, ocular administration, intra-aural administration, intranasal administration, inhalation, spraying via the mouth or nose, transdermal administration, percutaneous administration, or the like.

The pharmaceutical composition of the present invention is determined depending on the type of a drug, which is an active ingredient, along with various related factors such as a disease to be treated, administration route, the age, gender, and body weight of a patient, and the severity of diseases.

As used herein, the “subject” refers to a subject in need of treatment of a disease, and more specifically, refers to a mammal such as a human or a non-human primate, a mouse, a rat, a dog, a cat, a horse, and a cow, but the present invention is not limited thereto.

As used herein, the “administration” refers to providing a subject with a predetermined composition of the present invention by using an arbitrary appropriate method.

The term “prevention” as used herein means all actions that inhibit or delay the onset of a target disease. The term “treatment” as used herein means all actions that alleviate or beneficially change a target disease and abnormal metabolic symptoms caused thereby via administration of the pharmaceutical composition according to the present invention. The term “alleviation” as used herein means all actions that reduce the degree of parameters related to a target disease, e.g., symptoms via administration of the composition according to the present invention.

The present invention provides a method for screening a therapeutic agent for Alzheimer’s disease, the method comprising the following steps:
(a) treating a biological sample isolated from a patient with Alzheimer’s disease with a candidate material;
(b) measuring the activity level of PLCβ1 protein or the synthesis or secretion level of eCB in the sample; and
(c) determining the candidate material as a therapeutic agent for Alzheimer’s disease when the activity level of PLCβ1 protein or the synthesis or secretion level of eCB is increased compared to a group untreated with the candidate material.

In the present invention, the sample may be a hippocampus-derived sample, but is not limited thereto. Further, the measurement in Step (b) may be performed by one or more methods selected from the group consisting of western blot, enzyme linked immunosorbent assay (ELISA), radioimmunoassay (RIA), radioimmunodiffusion, and immunoprecipitation assay, but the method is not limited thereto.

In the present invention, the candidate material means an unknown material used for screening to examine whether the activity level of PLCβ1 protein or the synthesis or secretion level of eCB is recovered to the normal level. The candidate material includes a chemical, a peptide, a protein, an antibody and a natural extract, but is not limited thereto.

The present invention provides an information providing method for diagnosing Alzheimer’s disease, the method comprising measuring the activity level of PLCβ1 protein in a biological sample isolated from a subject.

In addition, the present invention provides a method for diagnosing Alzheimer’s disease, the method comprising measuring the activity level of PLCβ1 protein in a biological sample isolated from a subject.

Furthermore, the present invention provides a use of a composition comprising a PLC activator as an active ingredient for diagnosing Alzheimer’s disease.

Further, the present invention provides a use of a PLC activator for preparing a drug for diagnosing Alzheimer’s disease.

The method for diagnosing Alzheimer’s disease according to the present invention promptly diagnoses the disease, and thus enables immediate response such as administration of a therapeutic agent before the dementia worsens. Thus, the diagnostic method of the present invention may be effectively used to treat Alzheimer’s disease.

In the present invention, “diagnosis” includes determining the susceptibility of an object to a specific disease or disorder, determining whether an object currently has a specific disease or disorder, and determining the prognosis of an object with a specific disease or disorder.

In the present invention, the subject may be a mammal including a human, and may be a subject suspected of having Alzheimer’s disease, but is not limited thereto. In addition, the biological sample may be a hippocampus-derived sample, but is not limited thereto. Furthermore, the method may further comprise determining, as Alzheimer’s disease, the case where the measured activity level of the PLCβ1 protein is reduced compared to the activity level of the PLCβ1 protein of a control, but is not limited thereto. The control may be a biological sample derived from a normal animal, but is not limited thereto.

In the present invention, the measurement of the activity level of the PLCβ1 protein is not particularly limited as long as it is performed by a method for measuring the activity level of protein known in the art, but may be performed by, for example, one or more selected from the group consisting of western blot, enzyme linked immunosorbent assay (ELISA), radioimmunoassay (RIA), radioimmunodiffusion, and immunoprecipitation assay.

As used herein, “measurement” has a meaning including all of measuring and confirming whether a target material (a marker protein in the present invention) is present (expressed), or measuring and confirming a change in presence level (expression level) of a target material. That is, the measuring of the expression level of the protein means measuring whether the protein is expressed (that is, measuring the presence or absence of expression), or measuring the levels of qualitative and quantitative changes in the protein. The measurement may be performed without limitation by including all qualitative (analytical) and quantitative methods. The types of qualitative and quantitative methods in the measurement of the protein levels are well known in the art, and include the experimental methods described herein. Specific methods for comparing protein levels for each method are well known in the art. Therefore, the detection of the target protein means including detection of the presence or absence of cleaved PLCβ1, or confirmation of an increase (upregulation) or a decrease (downregulation) in the protein expression level.

In the present invention, the biological sample may be used without limitation as long as it is collected from a subject to be diagnosed with Alzheimer’s disease, may include, for example, tissue, cells, blood, serum, plasma, saliva, urine, and the like, and may be preferably measured by one or more selected from the group consisting of a biological tissue sample or cell sample, for example, cells derived from diseased tissue, tissue, organ, fine needle aspiration specimen, core needle biopsy specimen and vacuum aspiration biopsy specimen. The biological sample may be pretreated prior to use for detection or diagnosis. The pretreatment may include, for example, homogenization, filtration, distillation, extraction, concentration, inactivation of interfering components, addition of reagents, and the like. The above sample may be prepared to enhance the detection sensitivity of a protein marker, and for example, a sample obtained from a subject may be pretreated using a method such as anion exchange chromatography, affinity chromatography, size exclusion chromatography, liquid chromatography, sequential extraction or gel electrophoresis.

Further, the present invention may provide a method for treating Alzheimer’s disease, the method comprising the following steps:
(a) measuring the activity level of PLCβ1 protein in a biological sample isolated from a subject;
(b) determining, as Alzheimer’s disease, the case where the activity level of the PLCβ1 protein is reduced compared to the activity level of the PLCβ1 protein of a control; and
(c) treating Alzheimer’s disease.

In the present invention, as the treatment in Step (c), it is possible to use a method such as drug treatment, surgical treatment, cognitive rehabilitation treatment, and occupational treatment, but is not limited thereto.

In the present invention, the drug may be one or more drugs selected from the group consisting of Donepezil, Rivastigmine, Galantamine, memantine, and Tacrine-huperzine A, and may be an acetylcholinerase inhibitor or an NMDA receptor antagonist, but is not limited thereto.

In the present invention, the drug may be a pharmaceutical composition comprising the PLC activator according to the present invention as an active ingredient, but is not limited thereto.

Hereinafter, preferred examples for helping with understanding of the present invention will be suggested. However, the following examples are provided only so that the present invention may be more easily understood, and the content of the present invention is not limited by the following examples.

### Experimental Methods

### 1. Experimental Animal Model

To test the acute effect of AβO on hippocampal S-eCB mobilization, hippocampal slices from Sprague-Dawley rats (SD, 2 to 3 weeks old, DaeHan Biolink, Co., Ltd., Korea) were used. Male and female rats were used in all in vitro experiments. To test the chronic effect of AβO on hippocampus-dependent memory impairments caused by amyloidosis, the 5XFAD mouse model (6 to 7 months old, #34840-JAX, Jackson Laboratory, USA), which is a mouse model of Alzheimer’s disease that mimics AβO depositions, was used. C57BL/6 mice (6 to 7 months old, KOATECH, Korea) were used as wild type (WT) control mice for 5XFAD mice. Male 5XFAD and C57BL/6 mice were used for all in vivo behavioral experiments. The 5XFAD mouse genotypes were identified by polymerase chain reaction (PCR) using DNA extracted from the tails of the mice. All animal-related procedures followed the guidelines of the Institutional Animal Care and Use Committee of Korea University (guidelines for SD rats: KUIACUC-2017-103, guidelines for C57BL/6 and 5XFAD mice: KUIACUC-2019-0068).

### 2. Amyloid Beta Oligomer (AβO) Preparation

Soluble AβO was prepared according to a previous study (BMC Biol. 2020;18(1):7.). Specifically, Aβ1-42 (Aβ) and a scrambled form of Aβ as a control peptide were purchased in powder form. Aβ and scrambled Aβ were dissolved for monomerization in 1,1,1,3,3,3-hexafluoro-2-propanol (HFIP, Sigma Aldrich, USA) at a final concentration of 1 mM, and the solution was incubated for 90 minutes. After the HFIP was evaporated, the remaining thin and clear film of Aβ or scrambled Aβ was dissolved in dimethyl sulfoxide (DMSO, Sigma Aldrich, USA) to prepare 5 mM Aβ or scrambled Aβ stocks, which were aliquoted and frozen at -20° C. Aβ or scrambled Aβ stocks were thawed and diluted to 100 µM in artificial cerebrospinal fluid (ACSF). Thereafter, Aβ or scrambled Aβ stocks were incubated for oligomerization for 18 hours. The final AβO or scrambled AβO stock solution was diluted to a final concentration of 200 nM in 31.2 ml of ACSF, and rat hippocampal slices were treated with the stock solution for 20 minutes in a recovery chamber before measurement of brain signals. In control experiments for normal brain conditions using a vehicle, rat hippocampal slices were treated with 0.004% DMSO in ACSF.

### 3. Preparation of Ex Vivo Hippocampal Slices

The brains of SD rats were isolated following decapitation under isofluraneinduced anesthesia and transferred into ice-cold ACSF containing 126 mM NaCl, 3 KCl, 1.25 mM NaH₂PO₄, 2 mM MgSO₄, 2 mM CaCl₂, 25 mM NaHCO₃, and 10 mM glucose (pH 7.2 to 7.4). The ACSF was continuously oxygenated with 95% O₂/5% CO₂. Horizontal hippocampal slices (350 µm thickness) were obtained using a vibratome (VT1000S, Leica Biosystems, Germany) and immediately incubated at room temperature in a submerged chamber perfused with oxygenated ACSF for at least 1 hour.

### 4. Ex Vivo eCB Mobilization Protocols

To quantify eCB mobilization, the changes in eCB mobilization-induced Schaffer collateral (SC) nerve bundle stimulation-evoked inhibitory postsynaptic current (eIPSC) amplitudes were measured in CA1 PCs through the whole-cell voltage-clamp technique. The whole-cell voltage-clamp technique in CA1 PCs was performed using a MultiClamp 700B amplifier (Molecular Devices, USA) under an infrared differential interference contrast video microscope (BW51W, Olympus, Japan), using a borosilicate electrode (tip resistance: 4 to 8 MΩ) that was filled with an intracellular solution containing 145 mM CsCl, 10 mM HEPES, 2 mM EGTA, 4 mM QX-314, 4 mM Mg-ATP, 0.4 mM NaCl-GTP, pH 7.2 to 7.3, and 270 to 280 mOsm/L. To record eIPSCs in the CA1 PCs, a stimulating electrode (A-M Systems, USA) was placed in a stratum radiatum to stimulate the SC pathway that carries CA3 PC axons. Electrical stimulation pulses of 200 to 400 µA amplitudes (20 to 40 µs) were generated using a constant current stimulator (model DS3, Digitimer Ltd., UK). eIPSCs were recorded in CA PCs every 1 second while maintaining the membrane potential at -70 mV. Before the application of CB1R antagonist AM251, depolarization-induced suppression of inhibition (DSI) was induced by applying a voltage step from -70 mV to 0 mV for 1 second to detect cells sensitive to eCB.

To induce S-eCB mobilization, Group 1 mGluR agonist (S)-3,5-dihydroxyphenylglycine (DHPG, 50 µM) was locally injected using a puff pipette located near the somatic cells of CA1 PCs. To induce S-eCB mobilization, Group 1 mGluR activation was combined with postsynaptic depolarization. Further, a physiologically realistic postsynaptic depolarization was induced by mimicking in vivo-like sparse CA1 PC neural spike (1 Hz) observed in vivo during memory processing. To this end, 5 ms-voltage steps (from -70 to +40 mV) were delivered at 1 Hz for 60 s. DSI was measured using a mean amplitude of the first ten consecutive eIPSCs following the neural spikes and/or DHPG puff. In all voltage-clamp recordings, 6-cyano-7-nitroquinoxaline-2,3-dione (CNQX, 20 µM) and D-2-amino-5-phosphonovaleric acid (D-AP5, 50 µM) were applied to prevent ionotropic glutamatergic currents and synaptic plasticity. To test Group 1 mGluR subtypedependence of PLCβ-dependent S-eCB mobilization, the experiments were repeated in the presence of an mGluR1 antagonist, LY367385 (100 µM), or an mGluR5 antagonist, 2-methyl-6-(phenylethynyl) pyridine (MPEP, 10 µM). All data were filtered at 2 kHz and digitized at 5 kHz using an ITC-18 AD board (HEKA Elektronik, Germany). In addition, Igor Pro software (WaveMetrics, USA) was used to generate command signals and analyze the data.

### 5. S-eCB Mobilization Induced Spike Timing-Dependent Potentiation (tLTP) Protocol

To directly test whether S-eCB mobilization can induce hippocampal tLTP, the PLCβ-dependent S-eCB mobilization experimental protocol was modified by introducing presynaptic CA3 PC neural spikes during the S-eCB mobilization protocol to induce tLTP. Whole-cell current-clamp recordings were made from hippocampal CA1 PCs using a glass electrode filled with an intracellular solution containing 110 mM potassium gluconate, 4 mM NaCl, 40 mM HEPES, 4 mM Mg-ATP, 0.3 mM NaCl-GTP, and 270-280 mOsm/L, at pH of 7.2 to 7.3. Two stimulating electrodes were positioned on the stratum radiatum of the hippocampal CA1 region. One was used for monitoring an excitatory postsynaptic potential (EPSP) in the test pathway and the other was used for monitoring the EPSP in the control pathway. The tLTP induction protocol was implemented only in the test pathway. To directly test how S-eCB mobilization is related to tLTP induction, the tLTP induction paradigm was designed by modifying the S-eCB mobilization protocol. SC stimulation-evoked presynaptic CA3 PC neural spikes were introduced during the S-eCB mobilization protocol. 1 Hz presynaptic CA3 PC neural spikes were evoked 10 ms before the 1 Hz postsynaptic CA1 PC neural spikes during the activation of mGluR5 induced tLTP. This means that PLCβ-dependent S-eCB mobilization was induced during the pairing of presynaptic CA3 PC neural spikes and postsynaptic CA1 PC neural spikes for tLTP induction at the CA3-CA1 excitatory synapses. During the baseline EPSP recordings, SC stimulation-evoked EPSPs were evoked every 6 seconds for at least 10 minutes. After a stable baseline was established, tLTP was induced in which SC stimulation-evoked presynaptic CA3 PC neural spikes were paired with postsynaptic CA1 PC neural spikes evoked by current pulses (800 pA, 3 ms current steps) with a 10 ms time window at 1 Hz. This was repeated 200 times during the activation of mGluR5 through the application of DHPG (50 µM) in the presence of LY367385 (100 µM). SC stimulation-evoked EPSP responses were recorded for at least 30 minutes after the tLTP induction protocol. In all tLTP induction experiments, the membrane potentials of the CA1 PCs were maintained at -70 mV. To analyze the changes in synaptic efficacy following tLTP induction, an EPSP slope was measured using a linear fit on the rising slope of the EPSP between 20 to 25% and 75 to 80% in the EPSP peak amplitude during the baseline conditions. Changes in synaptic efficacy by the tLTP induction protocol were estimated as percentage changes relative to the mean EPSP slope of the 10-min baseline. To compare the synaptic efficacy changes according to various neurons and experimental conditions, the mean of the normalized EPSP slopes was used during the time period of 25 to 30 minutes after the end of the tLTP induction protocol.

### 6. Western Blotting

Hippocampal PLCβ1 of SD rats and C57BL/6 or 5XFAD mice were prepared from homogenized hippocampi dissolved in 200 µL of RIPA buffer (Bio-Rad, USA), and were resolved on non-reducing 10% tris-glycine-sodium dodecyl sulfate-polyacrylamide gel electrophoresis (SDS-PAGE) gels (Bio-Rad, USA) using 4× Laemmli sample buffers (Bio-Rad, USA). Thereafter, the gels were transferred onto 0.2-µm PVDF membranes (Bio-Rad, USA) according to the manufacturer’s recommendations. The membranes were blocked in 5% bovine serum albumin (BSA) in Tris-buffered saline containing 0.01% Tween 20 (TBST, Bio-Rad, USA) at room temperature for 1 hour. Blots were incubated with a primary antibody of PLCβ1 (0.4 µg/mL, NBP2-38220, Novus, USA) in a wash buffer containing 5% BSA at 4° C. overnight. Thereafter, the membranes were washed three times with TBST buffer. Then, the membranes were incubated with secondary horseradish peroxidase (HRP)-conjugated anti-rabbit antibodies (1:3000, Cat# 170-6515, Control# 64170140, RRID: AB_2617112, Bio-Rad, USA) in the wash buffer containing 5% BSA at room temperature for 1 hour. Immunoreactivity was confirmed with enhanced chemiluminescence (Bio-Rad, USA) using a Fluorchem E system (ProteinSimple, USA) and analyzed with ImageJ software. Molecular weight values were estimated using the Precision Plus Protein™ Dual Color Standards (Bio-Rad, USA).

### 7. Stereotaxic Drug Injection

For stereotaxic drug injections, C57BL/6 or 5XFAD mice were deeply anesthetized with 2% isoflurane (2 mL/min flow rate) and head-fixed into a stereotaxic frame (Stoelting Co., USA). Craniotomies were performed at four sites to target the CA1 regions of the ventral and dorsal hippocampi. This is for injecting each 0.5 µl of DMSO (0.05%) as a vehicle or m-3M3FBS (50 µM) as a PLC activator, at a rate of 0.1 µl/min through a Hamilton syringe using a motorized stereotaxic injector (Stoetling Co., USA). After the syringe was left in the brain for 5 minutes or more to allow for drug diffusion, the scalp was sutured and disinfected with antibiotics. After mice were returned to their home cage for recovery for three days, they underwent contextual fear conditioning.

### 8. Contextual Fear Conditioning (CFC) Protocol

CFC was performed in conditioning chambers (Coulbourn Instruments, USA) consisting of metal panel sidewalls and Plexiglas front and rear walls, and a stainless-steel grid floor consisting of 16 grid bars. The grid floor was connected to a precision animal shocker (Coulbourn Instruments, USA) set to deliver a 0.5 mA foot shock for 1 second. A ceiling-mounted video camera recorded the behavior activity, and fed the corresponding video into a customized computer software (MATLAB, USA). The chambers were washed with a 70% ethanol solution prior to animal placement. On day 1, C57BL/6 or 5XFAD mice were placed in the conditioning chamber for 20 minutes for free exploration (habituation). On day 2, C57BL/6 or 5XFAD mice were placed into the conditioning chamber for the fear conditioning session (483 seconds) consisting of three 0.5 mA 1-s-long foot shocks during a 120-second baseline period (interstimulus interval equal to 120 seconds; conditioning). On day 3, the mice were placed back into the conditioning chamber for 2 min to evaluate their memory in an electric shock-free state. Behavioral mobility data collected during the contextual conditioning experiments were automatically detected using the gray scaling method of EthoVision XT (Noldus, USA). This method used light and dark thresholds to determine a subject in the conditioning chamber. Since a body-freezing response (freezing) is considered a conditioned response to CFC during the memory session, the body-freezing response was detected when the threshold was below the 2.0% threshold of mobility. That is, this means that there could be no more than a 2.0% change in the pixels of a detected mouse between the current sample and the previous sample. The freezing ratio was estimated by scoring a freezing behavior defined as the absence of movement for 2 seconds or more, except that it required respiration.

### 9. Drugs

Group 1 mGluR agonist DHPG (50 µM, Tocris, UK) was used to activate Group 1 mGluR. To block mGluR5 and mGluR1a, MPEP (10 µM, Tocris, UK) and LY367385 (100 µM, Tocris, UK) was applied to rat hippocampal slices, respectively. AM251 (3 µM, Tocris, UK) was used to block presynaptic CB1R. U73122 (5 µM, Tocris, UK) was used to block PLCβ activity. m-3M3FBS (30 µM, Tocris, UK) was used as a PLC activator. An NMDAR antagonist D-AP5 (50 µM, Tocris, UK) and an AMPA receptor antagonist CNQX (20 µM, Tocris, UK) were used for the eIPSC recordings. AβO and scrambled AβO were synthesized from a lyophilized powder of Aβ and scrambled Aβ peptide, respectively (Bachem, Japan). A 4× Laemmli sample buffer (Bio-Rad, USA) and running buffer (Bio-Rad, USA) were used for western blot SDS-PAGE. In the case of the antibody incubation step in western blotting, a rabbit monoclonal primary antibody (NBP2-38220, Novus, USA) and HRP-conjugated secondary anti-rabbit antibodies (Cat# 170-6515, Control# 64170140, RRID: AB_2617112, Bio-Rad, USA) were used.

### 10. Statistical Analysis

All data are expressed as mean ± standard error of the mean (SEM). Statistical significance was measured using Student’s t test or one-way analysis of variance (ANOVA) followed by a post hoc Tukey’s test. Statistical significance was set at p < 0.05.

### Example 1. Confirmation of Effects of AβO Suppressing Synergistic Enhancement of eCB (S-eCB) Mobilization in Hippocampal CA1 Pyramidal Cells

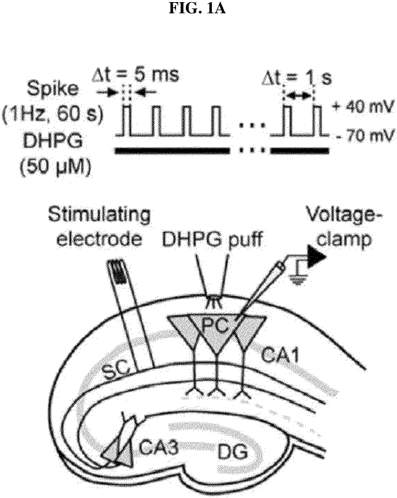

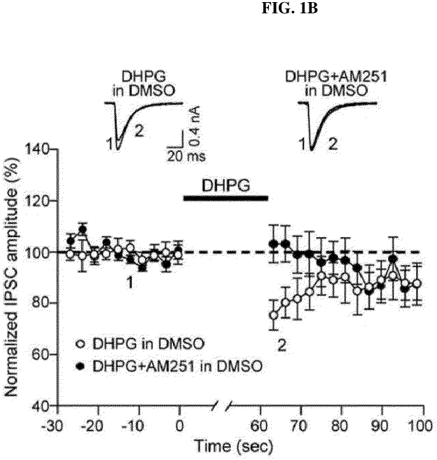

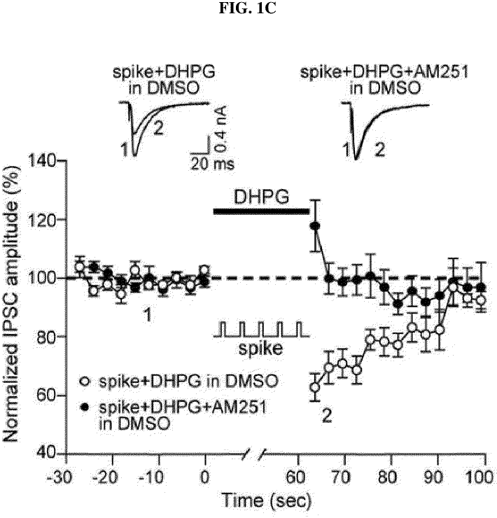

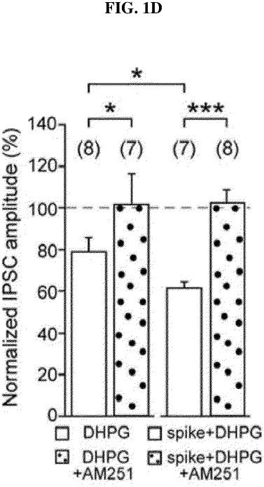

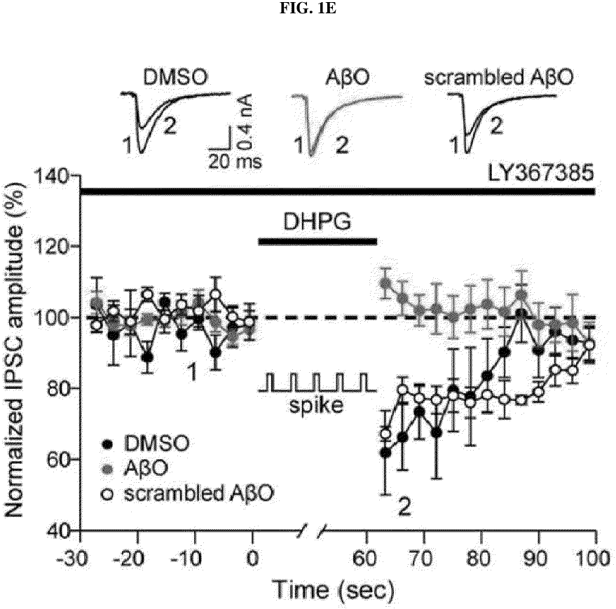

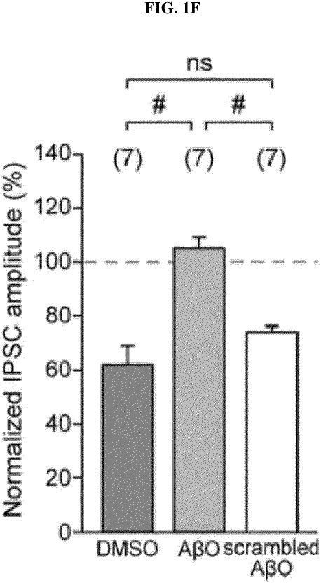

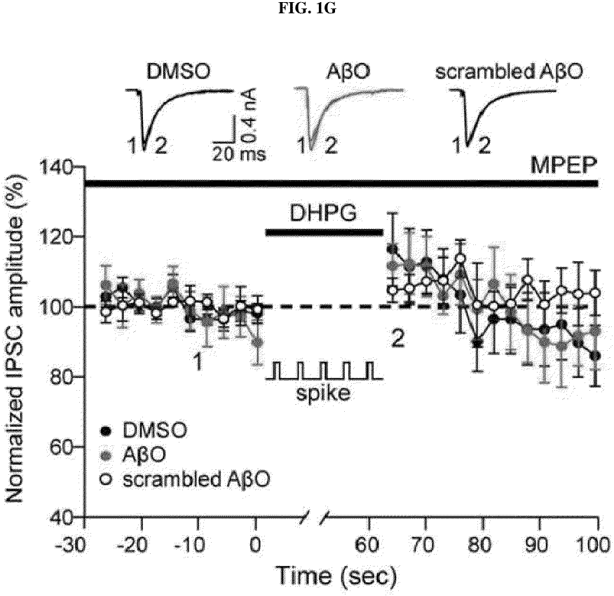

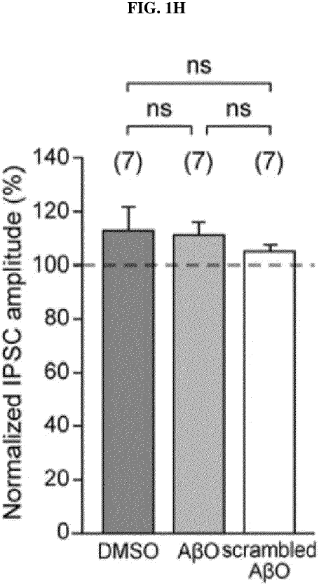

To investigate the effect of AβO on PLCβ-dependent eCB and S-eCB mobilization in CA1 PCs, a protocol capable of measuring S-eCB mobilization induced by a co-activation of Group 1 mGluR during postsynaptic depolarization was established. Whole-cell voltage-clamp recordings were performed in CA1 PCs from rat hippocampal slices, and eIPSC amplitudes were recorded, respectively, before and after activation of Group 1 mGluR using the Group 1 mGluR agonist DHPG (50 µM), or with co-activation of Group 1 mGluR with postsynaptic depolarization. The results are illustrated in FIGS. 1A to 1D.

As illustrated in FIGS. 1B and 1D, DHPG puff alone induced the suppression of the eIPSC amplitudes, and the first ten eIPSC amplitudes decreased to 79 ± 7% of the mean eIPSC amplitude measured at the baseline before the DHPG puff. Further, treatment with the CB1R antagonist AM251 (3 µM) blocked the decrease in amplitude. This indicates that the activation of Group 1 mGluR induces eCB mobilization.

Next, it was confirmed whether pairing DHPG puff with 60-s-long in vivo-like postsynaptic neural spike trains at 1 Hz could induce S-eCB mobilization in DMSO-treated rat hippocampal slices. The results are illustrated in FIGS. 1C and 1D.

As illustrated in FIGS. 1C and 1D, eIPSC amplitudes were completely blocked by AM251, and thus suppressed to 62 ± 4% of the baseline eIPSC amplitude. In fact, DHPG puff during in vivo-like neural spike trains induced synergistic enhancement in the suppression of eIPSC amplitudes. This indicates that the in vivo-like neural spike trains during Group 1 mGluR activation can induce S-eCB mobilization.

Group 1 mGluR consists of two subtypes in the hippocampus, mGluR1 and mGluR5. Therefore, to confirm whether the subtypes of Group 1 mGluR mainly induce S-eCB mobilization, DMSO-treated, AβO-treated, and scrambled AβO-treated rat hippocampal slices were treated with an mGluR1 antagonist, LY367385 (100 µM), or an mGluR5 antagonist, MPEP (10 µM), and the S-eCB mobilization experiment was repeated. The results are illustrated in FIGS. 1E to 1H.

As illustrated in FIGS. 1E to 1H, in the presence of LY367385, the eIPSC amplitudes following 60-s-long DHPG puff during 1 Hz neural spikes decreased in both DMSO-treated rat hippocampal slices and scrambled AβO-treated rat hippocampal slices. However, the suppression of eIPSC amplitudes was completely blocked in AβO-treated rat hippocampal slices, which was remarkably different from those recorded in DMSO-treated rat hippocampal slices and scrambled AβO-treated rat hippocampal slices. In the presence of MPEP, the eIPSC amplitudes following DHPG puff during 1 Hz neural spikes did not significantly change in the DMSO-treated, AβO-treated, or scrambled AβO-treated rat hippocampal slices.

The above results indicate that in vivo-like neural spike trains during the activation of mGluR5, but not mGluR1, can induce S-eCB mobilization. In addition, it was confirmed that such S-eCB mobilization is impaired specifically by AβO as scrambled AβO suppresses S-eCB mobilization.

### Example 2. Confirmation of Effects in Which AβO Reduces PLCβ to Impair S-eCB Mobilization in Hippocampal CA1 Pyramidal Cells

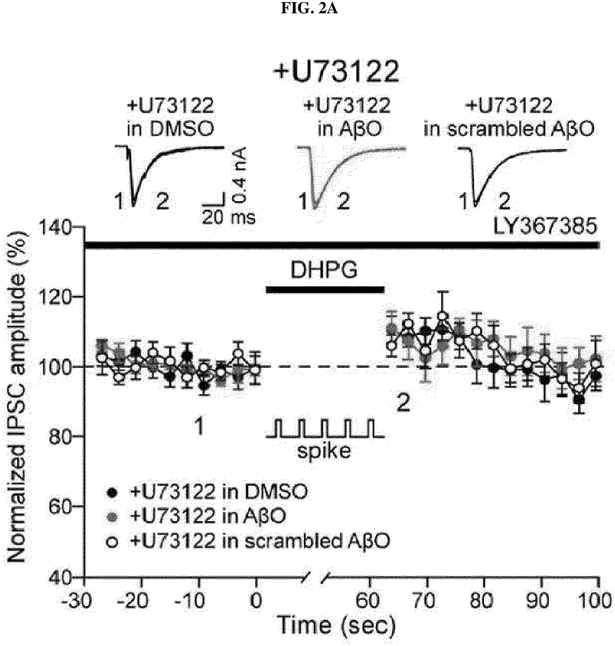

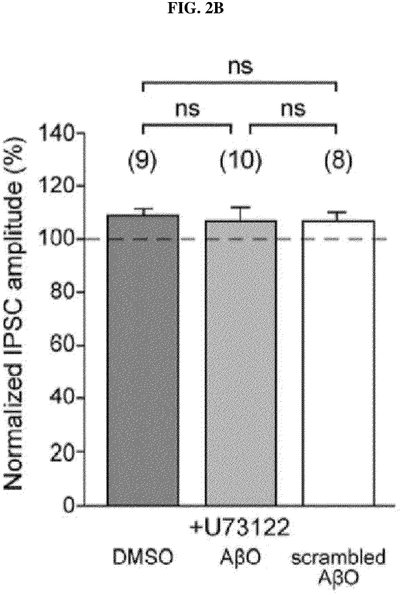

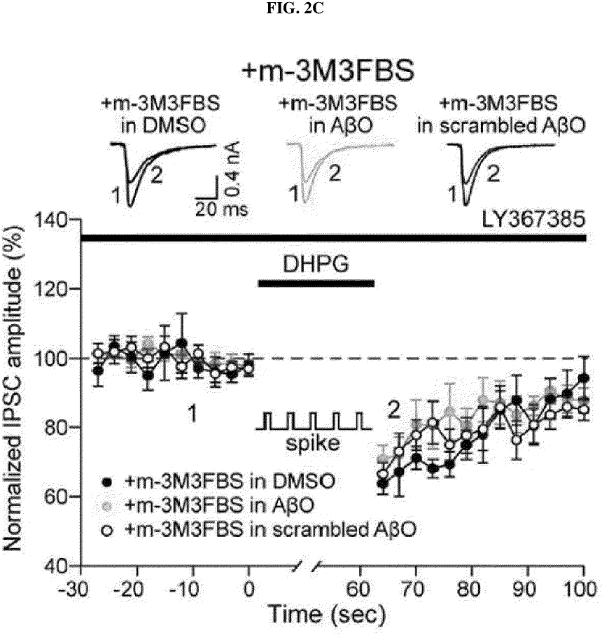

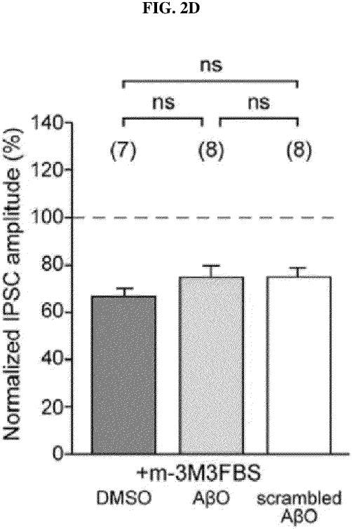

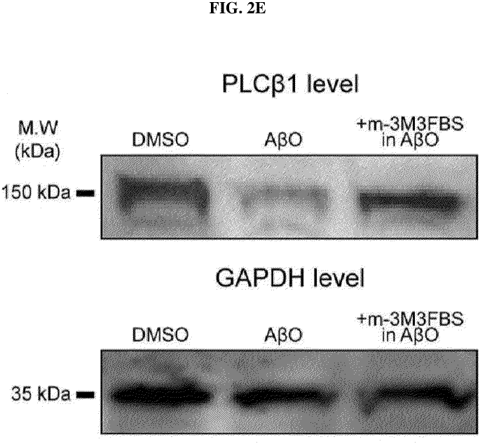

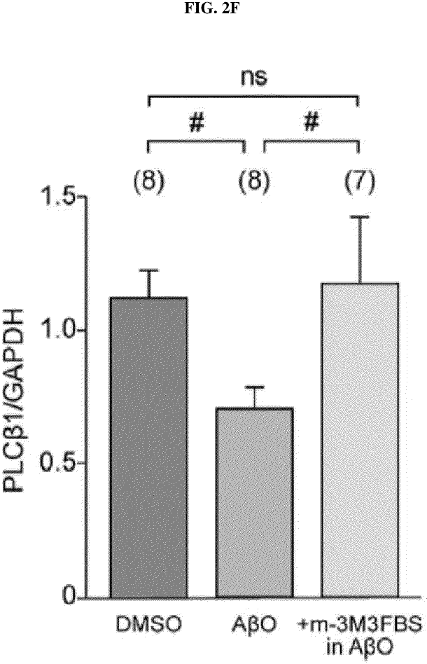

PLCβ isozymes that are involved in eCB mobilization act as sensors for detecting the co-activation of postsynaptic depolarization and mGluR5 activation. Considering that S-eCB mobilization is suppressed by AβO, a downstream chemical cascade that is involved in PLCβ or eCB production may be affected by AβO. To confirm this, a PLCβ blocker, U73122 (5 µM) was applied, and the S-eCB mobilization experiment was repeated in DMSO-treated, AβO-treated, and scrambled AβO-treated rat hippocampal slices. The results are illustrated in FIGS. 2A and 2B.

As illustrated in FIGS. 2A and 2B, it was confirmed that U73122 completely blocked the synergistic enhancement in the suppression of eIPSC amplitudes in DMSO-treated rat hippocampal slices. Furthermore, the patterns were also similar to those in the AβO-treated and scrambled AβO-treated rat hippocampal slices. This indicates that S-eCB mobilization suppressed by AβO is completely blocked by U73122, meaning that AβO can block S-eCB mobilization in CA1 PCs by interfering with the PLCβ pathway.

Since a decrease in PLCβ interferes with S-eCB mobilization in AβO-treated rat hippocampal slices, it was hypothesized that an increase in PLCβ activity using a PLC activator, m-3M3FBS, would be able to recover AβO-induced impairment of S-eCB mobilization. To test this hypothesis, DMSO-treated, AβO-treated, and scrambled AβO-treated rat hippocampal slices were treated with m-3M3FBS (30 µM), and the S-eCB mobilization experiment was repeated. The results are illustrated in FIGS. 2C and 2D.

As illustrated in FIGS. 2C and 2D, in the presence of m-3M3FBS, synergistic enhancement of the suppression of eIPSC amplitudes could be completely recovered in the AβO-treated rat hippocampal slices. Recordings in DMSO-treated and scrambled AβO-treated rat hippocampal slices were also not significantly different. This indicates that PLCβ is impaired by AβO to interfere with S-eCB mobilization, and increasing PLC activation using m-3M3FBS can restore S-eCB mobilization suppressed by AβO. To confirm that the reduction in hippocampal PLCβ (particularly, PLCβ1, which is enriched in the hippocampus and is known to be involved in mGluR-induced eCB mobilization) protein expression is mediated by AβO, western blot was performed. These are illustrated in FIGS. 2E and 2F.

As illustrated in FIGS. 2E and 2F, the protein expression levels of PLCβ1 in the AβO-treated rat hippocampal slices were remarkably decreased compared to the DMSO-treated rat hippocampal slices. However, treatment with m-3M3FBS completely restored the PLCβ1 protein levels in the AβO-treated rat hippocampal slices, similar to the results in the DMSO-treated rat hippocampal slices. The above results mean that AβO suppresses PLCβ-dependent eCB mobilization by directly reducing the protein expression levels of PLCβ1.

### Example 3. Confirmation of Effects in Which Activation of PLCB Restores AβO-Induced Impairment of Spike-Timing-Dependent Potentiation in Hippocampal CA3-CA1 Synapses

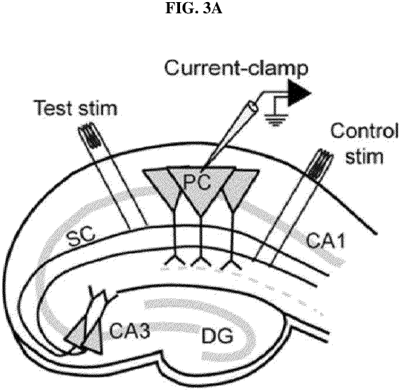

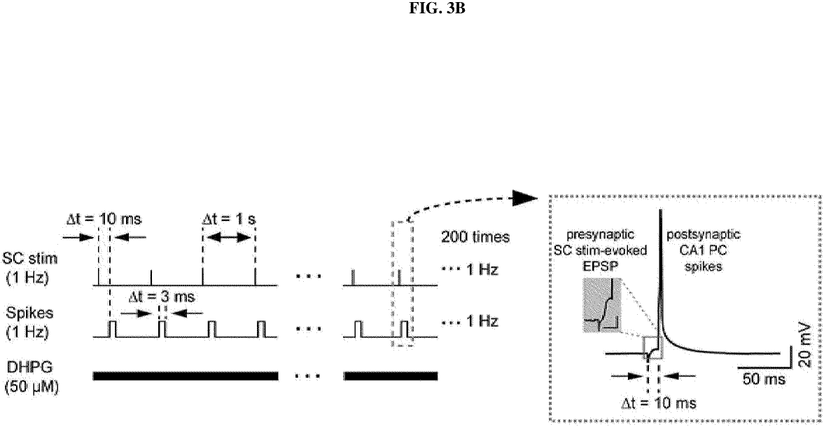

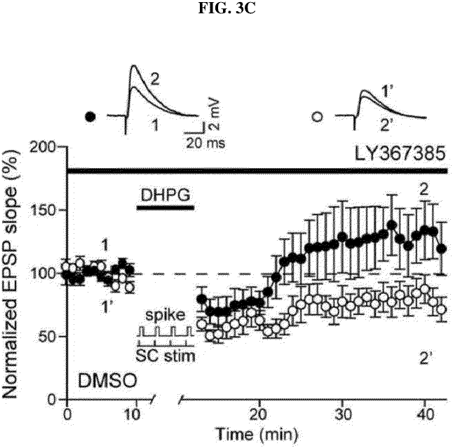

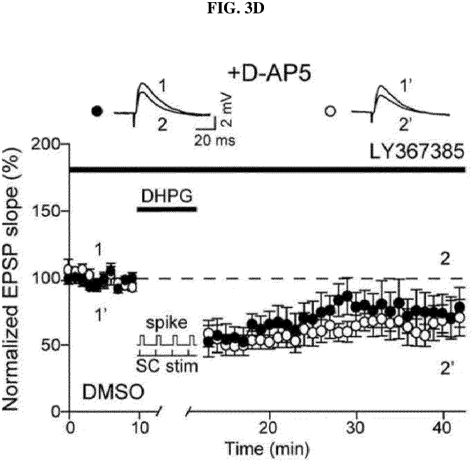

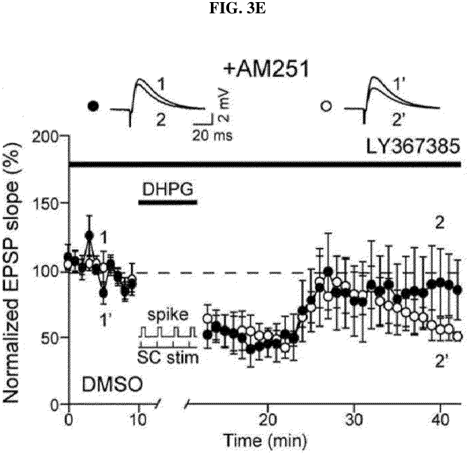

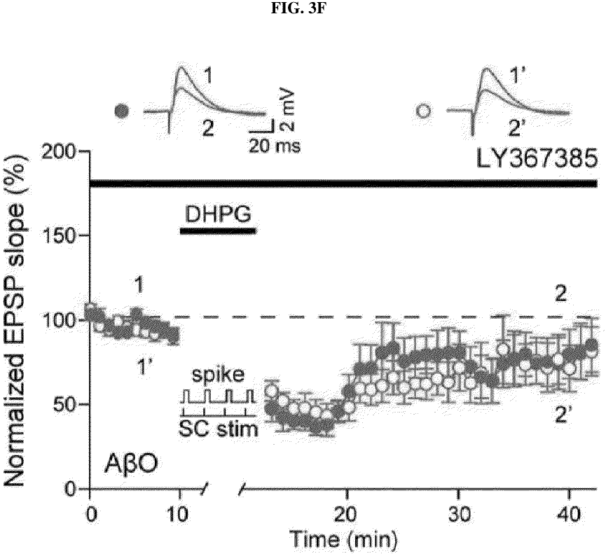

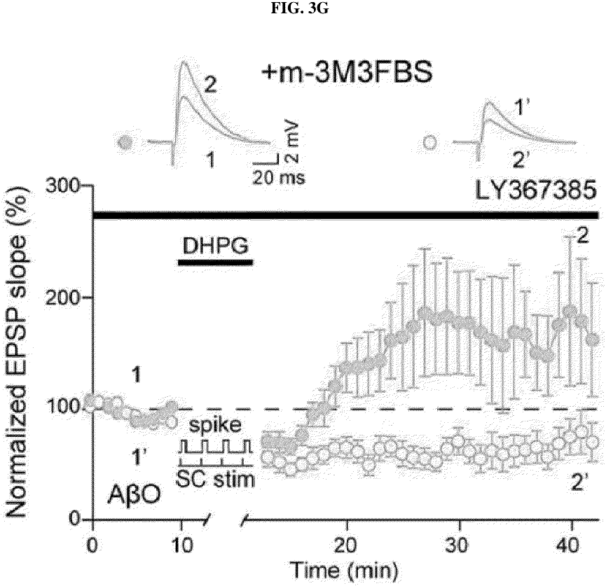

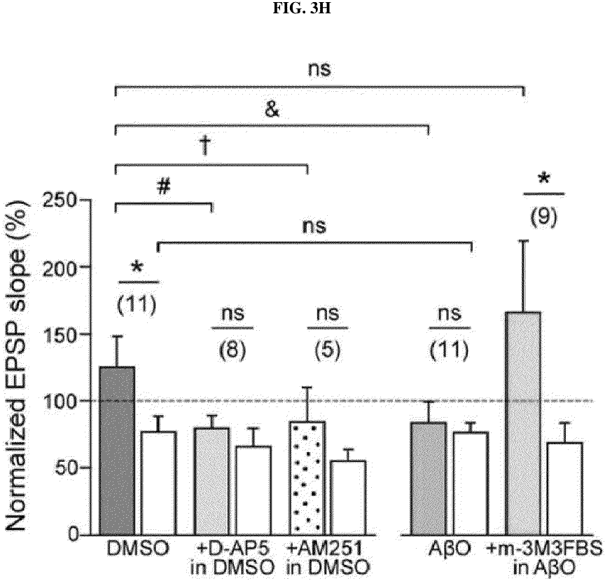

To confirm whether there is a causal relationship between suppression of S-eCB mobilization induced by AβO and impairment of LTP induction by AβO, presynaptic CA3 PC neural spikes were introduced during the S-eCB mobilization protocol. This ensured S-eCB mobilization while presynaptic CA1 PC and CA3 PC neural spikes were paired at a 10 ms time window to induce tLTP. Further, the S-eCB mobilization-ensuring tLTP protocol induced the remarkable increase in EPSP slopes in the test pathway compared to the control pathway. The results are illustrated in FIGS. 3A to 3H.

As illustrated in FIGS. 3A to 3H, mGluR5-mediated long-term depression (LTD) was induced in the control pathway, meaning that the activation of mGluR5 induces LTD at the CA3-CA1 synapse. tLTP was completely blocked by D-AP5 (50 µM) (FIG. 3D) and AM251 (3 µM) (FIG. 3E) whereas mGuR5-mediated LTD was induced in both the test and control pathways. This indicates that mGluR5-mediated LTD is independent of N-methyl-D-aspartate receptor (NMDAR) and Cannabinoid type 1 receptor (CB1R), whereas S-eCB mobilization is required for the induction of NMDAR-dependent tLTP.

When the tLTP induction was repeated in AβO-treated rat hippocampal slices, it was confirmed that AβO completely blocked the induction of tLTP in the test pathway (FIGS. 3F and 3H). In contrast, mGluR-LTD in the control pathway was not affected by AβO when compared to the control pathways in AβO-treated and DMSO-treated rat hippocampal slices (FIGS. 3C, 3F, and 3H). This means that while sparing mGluR5-mediated LTD, AβO specifically impaired NMDAR-dependent tLTP by interfering with S-eCB mobilization.

Since an increase in PLC activity recovered S-eCB mobilization, it was hypothesized that m-3M3FBS could also recover tLTP impaired by AβO. As a result of treating AβO-treated rat hippocampal slices with m-3M3FBS (30 µM), tLTP was completely recovered (FIGS. 3G and 3H). This is similar to the level observed in the DMSO-treated rat hippocampal slices. The above results mean that enhancing PLCβ-dependent S-eCB mobilization by directly increasing the PLCβ1 protein levels can recover hippocampal tLTP impaired by AβO. Therefore, it is confirmed that the PLC activator recovers hippocampal synaptic plasticity.

### Example 4. Confirmation of Effects in Which Activation of PLCβ Recovers Hippocampal PLCβ1 Protein Levels and Contextual Fear Memory in 5XFAD Mice

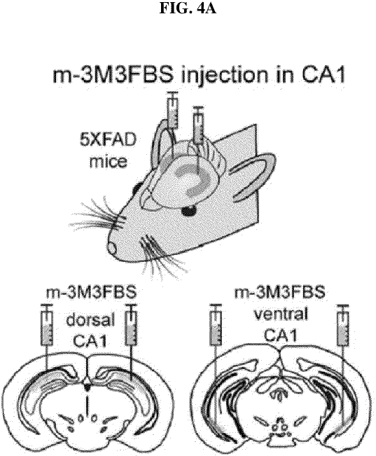

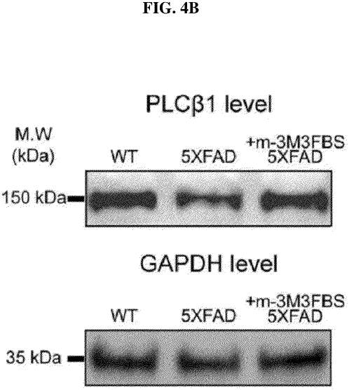

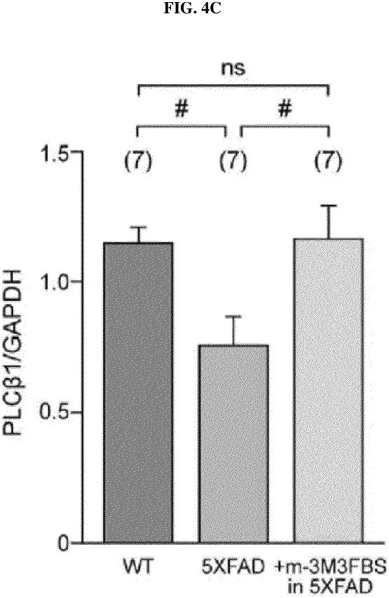

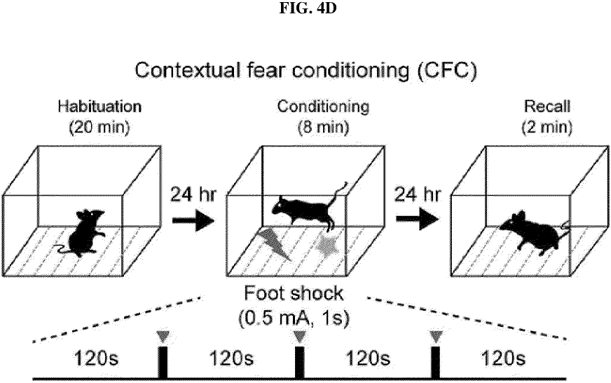

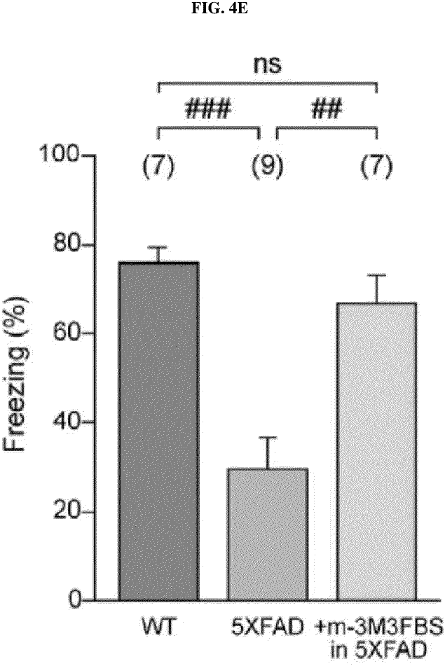

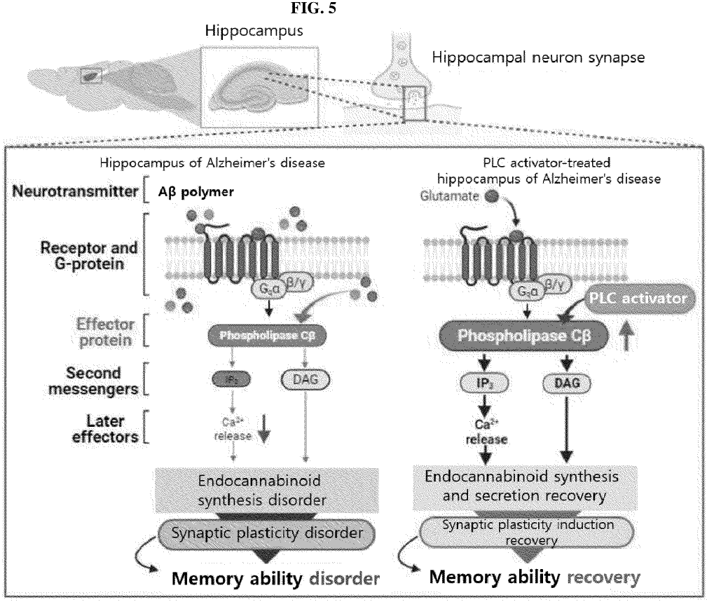

Considering that an increase in the PLCβ1 protein levels can recover AβO-impaired tLTP, and that tLTP is a synaptic mechanism underlying learning and memory, it was hypothesized that an increase in the PLCβ1 protein levels could recover behavioral memory impairment in Alzheimer’s disease. An Alzheimer’s disease 5XFAD mouse model known as a representative rodent model that mimics AβO pathophysiology in Alzheimer’s disease was used to confirm whether PLCβ1 protein levels are affected in 5XFAD mice and m-3M3FBS was pharmacologically was used to confirm whether behavioral memory impairment in Alzheimer’s disease can be recovered. In addition, it was confirmed whether impaired contextual fear memory could be restored by PLCβ activation in 5XFAD mice. The results are illustrated in FIGS. 4A to 4E. Furthermore, the recovery mechanism of synaptic plasticity impairment and memory ability disorder by PLC activators in the hippocampus of Alzheimer’s disease is illustrated in FIG. 5.

As illustrated in FIG. 4A, DMSO was injected into WT mice (C57BL/6) and 5XFAD mice, or m-3M3FBS was injected into both the dorsal and ventral regions of the bilateral hippocampi of 5XFAD mice.

Three days after injection, western blot analyses of PLCβ1 were performed. As illustrated in FIGS. 4B and 4C, the PLCβ1 protein levels in mouse hippocampal slices from DMSO-injected 5XFAD mice were remarkably decreased compared to those from DMSO-injected WT mice. This is consistent with the case of treatment with AβO. Meanwhile, it can be confirmed that the PLCβ1 protein level in hippocampal slices cut from 5XFAD mice injected with m-3M3FBS was recovered. Therefore, hippocampal PLCβ1 protein levels were significantly decreased not only in hippocampal slices of AβO-treated mice but also in the hippocampi of 5XFAD mice at the chronic stage of AD. Further, it was confirmed that m-3M3FBS completely recovered PLCβ1 protein levels back to normal.

To confirm whether a decrease in PLCβ1 protein levels correlates with hippocampus-dependent memory impairment in a mouse model of Alzheimer’s disease, a contextual fear memory experiment was performed in DMSO-injected WT, DMSO-injected 5XFAD, and m-3M3FBS-injected 5XFAD mice. The results are illustrated in FIGS. 4D and 4E.

As illustrated in FIGS. 4D and 4E, the freezing response in 5XFAD mice during memory recall was significantly lower than that in DMSO-injected WT mice. Meanwhile, it was confirmed whether a PLC activator could recover contextual fear memory disorder in m-3M3FBS-injected 5XFAD mice. As a result, m-3M3FBS-injected 5XFAD mice showed remarkably increased freezing responses compared to DMSO-injected 5XFAD mice. This means that PLCβ1 protein levels are related to successful memory recall and an increase in PLCβ1 protein levels can recover contextual fear memory impairment in AD mice.

Furthermore, as illustrated in FIG. 5, it was confirmed that long-term synaptic plasticity was recovered by recovering the PLC-eCB synthesis and secretory pathways induced by PLC activators, and the impairment of fear memory was also recovered to the normal level.

A composition comprising the PLC activator of the present invention as an active ingredient restores the S-eCB mobilization suppressed by AβO, recovers the synaptic plasticity impaired by AβO, and not only recovers PLCβ1 protein levels to normal levels in AβO-treated mouse hippocampal slices and 5XFAD mouse hippocampal slices in the chronic stage of AD, but also recovers contextual fear memory impairment in AD mice, and thus is expected to be usefully used for preventing or treating Alzheimer’s disease.

The above-described description of the present invention is provided for illustrative purposes, and those skilled in the art to which the present invention pertains will understand that the present invention can be easily modified into other specific forms without changing the technical spirit or essential features of the present invention. Therefore, it should be understood that the above-described Examples are illustrative only in all aspects and are not restrictive.

What is claimed is:

1. A method for treating or alleviating Alzheimer’s disease, the method comprising administering a composition comprising a phospholipase C (PLC) activator as an active ingredient to a subject in need.

2. The method of claim 1, wherein the PLC activator increases the activity of PLCβ1 protein.

3. The method of claim 1, wherein the PLC activator increases the synthesis and secretion of endocannabinoid (eCB).

4. The method of claim 1, wherein the PLC activator recovers any one or more of the following characteristics:
(a) hippocampal synaptic plasticity; and
(b) contextual fear memory.

5. The method of claim 1, wherein the PLC activator is m-3M3FBS.

6. A method for screening a therapeutic agent for Alzheimer’s disease, the method comprising the following steps:
(a) treating a biological sample isolated from a patient with Alzheimer’s disease with a candidate material;
(b) measuring the activity level of PLCβ1 protein or the synthesis or secretion level of eCB in the sample; and
(c) determining the candidate material as a therapeutic agent for Alzheimer’s disease when the activity level of PLCβ1 protein or the synthesis or secretion level of eCB is increased compared to a group untreated with the candidate material.

7. The method of claim 6, wherein the sample is a hippocampus-derived sample.

8. The method of claim 6, wherein the measurement in Step (b) is performed by one or more methods selected from the group consisting of western blot, enzyme linked immunosorbent assay (ELISA), radioimmunoassay (RIA), radioimmunodiffusion, and immunoprecipitation assay.

9. A method for diagnosing Alzheimer’s disease, the method comprising measuring the activity level of PLCβ1 protein in a biological sample isolated from a subject.

10. The method of claim 9, wherein the subject is a mammal comprising a human.

11. The method of claim 9, wherein the biological sample is a hippocampus-derived sample.

12. The method of claim 9, further comprising determining, as Alzheimer’s disease, a case wherein the measured activity level of the PLCβ1 protein is reduced compared to the activity level of the PLCβ1 protein of a control.

13. The method of claim 9, wherein the activity level of PLCβ1 protein is measured by one or more methods selected from the group consisting of western blot, enzyme linked immunosorbent assay (ELISA), radioimmunoassay (RIA), radioimmunodiffusion, and immunoprecipitation assay.
